(19) United States
(12) Patent Application Publication
(10) Pub. No.: US 2026/0317842 A1
(43) Pub. Date: Oct. 15, 2026

(54) COMPOSITIONS, KITS, AND METHODS FOR DETECTION OF VARIANT STRAINS OF AFRICAN SWINE FEVER VIRUS

(71) Applicant: Life Technologies Corporation, Carlsbad, CA (US)

(72) Inventors: Sandrine Moine, Saint-Trivier-sur-Moignans (FR); Elise Martin, Messimy-sur-Saône (FR); Aurore Blanc, Villefranche-sur-Saône (FR)

(21) Appl. No.: 19/101,229

(22) PCT Filed: Jul. 28, 2023
(86) PCT No.: PCT/US2023/029005

§ 371 Date: Feb. 4, 2025

Related U.S. Application Data
(60) Provisional application No. 63/395,667, filed on Aug. 5, 2022.

(51) Int. Cl. C12Q 1/70; C12Q 1/6851

(52) U.S. Cl. CPC C12Q 1/701; C12Q 1/6851; C12Q 2600/16; C12Q 2600/166

(57) ABSTRACT

Disclosed are compositions, methods, systems, and kits for the detection of African swine fever virus (ASFV) in a test sample, and in particular for distinguishing between wild/reference type ASFV and mutant/variant strains of ASFV. A variant ASFV assay includes a first set of primers and a first probe that correspond to a first ASFV target, and a second set of primers and a second probe that correspond to a second ASFV target. The first and second probes are differentially labelled. The first ASFV target is a MGF360 gene and the second ASFV target is the CD2v gene. Absence of these targets, in conjunction with a positive determination for another generic ASFV target such as the p72 gene, is indicative of a vaccine-associated variant strain of ASFV.

### CROSS REFERENCE TO RELATED APPLICATIONS

This application is a 371 U.S. National Phase Application off of International Application number PCT/US2023/029005, filed Jul. 28, 2023, which claims priority to U.S. Application Ser. No. 63/395,667, filed on Aug. 5, 2022.

### INCORPORATION BY REFERENCE OF SEQUENCE LISTING

The present application is being filed with a Sequence Listing in electronic format. The Sequence Listing is provided as a file entitled TP346835USPCT Sequence Listing.xml, created on Sep. 26, 2023, and is 8,861 bytes in size. The information in electronic format of the Sequence Listing is incorporated by reference in its entirety

### FIELD

The present teachings relate to compositions, methods, systems, and kits for the detection of African swine fever virus (ASFV) in a test sample, and in particular, for distinguishing between wild/reference type ASFV and mutant/variant strains of ASFV.

### BACKGROUND

ASFV is a relatively large double-stranded DNA virus that causes African swine fever (ASF). ASF is associated with hemorrhagic fever and high mortality rates in domestic pigs. Even with milder infections, infected animals lose weight and often develop pneumonia, skin ulcers, and swollen joints. Pregnant sows that contract ASF will often undergo spontaneous abortion, or the infection will lead to stillbirths. ASF thus represents a serious challenge to domestic pig operations in several regions of the world. Even in regions that have not yet experienced an ASF outbreak, such as the United States, the risk of transmission and outbreak remains present.

Natively, ASFV passes from a soft tick that infects several types of wild African swine, including giant forest hogs, warthogs, and bushpigs. Infection is generally asymptomatic in wild hosts. However, as large-scale domestic pig operations began in Africa, ASFV passed to domestic pigs and ASF outbreaks have occurred in various regions since the early twentieth century. ASF may be spread by infected ticks, but most transmission of concern is caused by transmission between pigs. The virus may be transmitted through direct or indirect contact with infected pigs, their feces, or their body fluids. The virus also survives for multiple months or even years within pork products, so slaughtered pigs (from hunting or domestic production) can be a transmission vector.

Due to the significant economic risks associated with the disease, there have been attempts to create and implement vaccines against ASF. At some point in recent years, likely due to desperation and lack of other options, some pig operations in China began using an unofficial and unapproved ASF vaccine. The illegal vaccine was apparently intended to provide an attenuated form of the virus to promote immunity in vaccinated animals. However, although infections from the illegal vaccine appear to be somewhat milder than wild type infections, they are still associated with reduced weight, reduced offspring viability, and reproductive issues such as stillbirths, abortions, and infertility. Moreover, vaccinated animals shed the virus and pass the infection to others.

Veterinarians and livestock epidemiologists face significant challenges in diagnosing infected animals and monitoring spread of ASF. Efforts to monitor and contain outbreaks are burdened by the fact that both wild type ASFV and mutant/variant versions of ASFV are now present. Assays designed for wild type ASFV cannot distinguish between ASFV variants, and it can be difficult to determine whether ASF, even once detected in a population, is a result of wild type infection or from illegal vaccine activity and its subsequent transmission of variant ASFV. Those tasked with monitoring ASF are thus handicapped in their ability to accurately determine disease source, potential routes of transmission, and options for containing and preventing ASF outbreaks.

Accordingly, there is an ongoing need for improved ASF assays capable of distinguishing between wild type ASFV and variant ASFV associated with unapproved vaccines.

### BRIEF DESCRIPTION OF THE DRAWINGS

FIG. 1 illustrates a schematic of an ASFV genome to show example loci that can be targeted in order to detect ASFV and to distinguish between wild type and certain vaccine-associated variants.

FIG. 2 illustrates an example of two separate process flows (i.e., methods) for using a combined ASFV assay that includes a generic ASFV sub-assay with an IPC and a variant ASFV sub-assay without an IPC.

FIG. 3 illustrates an example of an external positive control (EPC) that may be utilized in conjunction with variant primer/probe sets (e.g., as in Table 1) in an assay for determining whether ASFV present in a sample is wild type or a variant.

### DETAILED DESCRIPTION

### Overview of ASF Assays

As discussed above, unapproved ASF vaccines began circulating in China in recent years. Whether or not such unapproved vaccines are still in use, the ASFV variants associated with such vaccines are still present and risk of transmission to new populations remains. At least one of the illicit vaccines, or perhaps several such vaccines, include a double deletion ASFV with deletion of the MGF360 and CD2v genes. Apparently, it was believed that deletion of these two genes would sufficiently attenuate the virus. However, as discussed above, animals infected with this double deletion variant still display undesirable symptoms, in particular with respect to reproductive issues.

Moreover, there are legitimate vaccine candidates that, although not yet approved, also include deletion of the MGF360 and CD2v genes. For example, one current proposal is to delete six different genes between MGF360 and MGF505R (between 27,942-35,500 bp) and CD2v (73,394-74,476 bp) based on the virulent strain Pig/CN/HLJ/2018 (GenBank Accession Number: MK333180) (genotype II). Such strains of ASFV would also include deletion of the MGF360 and CD2v genes.

Researchers, veterinarians, agricultural epidemiologists, pig operation managers, and other stakeholders have an ongoing interest in monitoring ASF, protecting against ASF outbreaks, and containing outbreaks as they occur. Accordingly, such stakeholders are in need of assays that can not only readily identify ASFV in samples, but are also capable of distinguishing between wild type ASFV and vaccine-associated ASFV variants. The ability to make such distinctions can aid in determining the source of an outbreak, the potential progression of an outbreak, the best steps to remedy and contain the outbreak, and the best preventative measures on which to focus to avoid similar outbreaks in the future. The ability to effectively distinguish between wild type ASFV and a vaccine-associated variant can also beneficially assist researchers who are attempting to make accurate epidemiological assessments based on animal symptoms and outcomes.

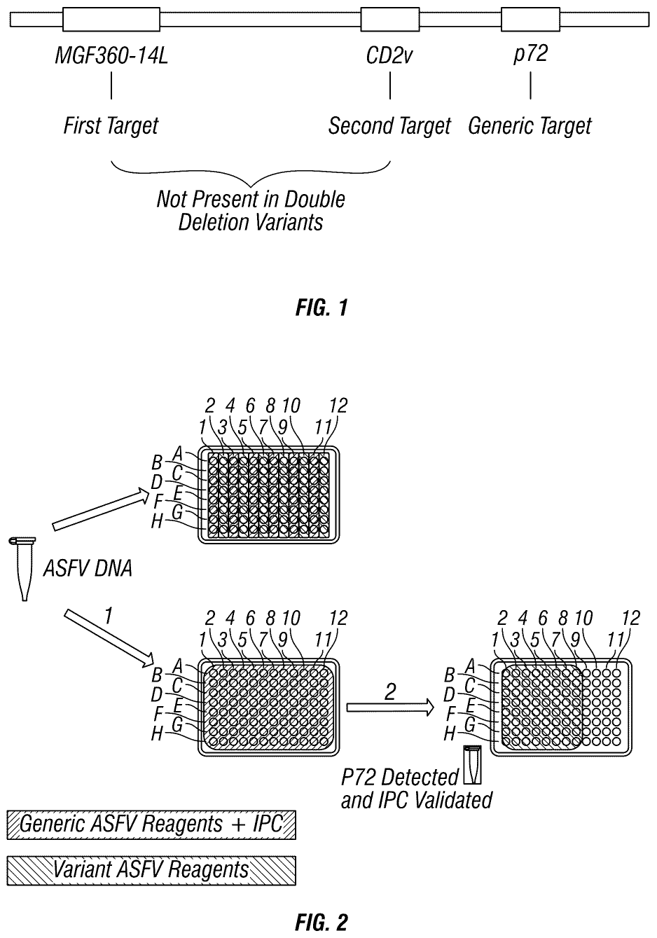

FIG. 1 illustrates a schematic of an ASFV genome to show example loci that can be targeted in order to detect ASFV and to distinguish between wild type and certain vaccine-associated variants. As show, a first target includes an MGF360 gene (e.g., the MGF360-14L gene), and a second target includes the CD2v gene. A generic target may also be included. FIG. 1 shows an example of such a generic target as the p72 gene.

Assays disclosed herein are designed to target such genes to enable detection of ASFV and to determine whether detected ASFV is wild type or is likely to be a vaccine-associated variant. Continuing with the example shown in FIG. 1, detection of the generic target (e.g., the p72 target) enables a determination that the sample is positive for ASFV. One or more of the other targets may additionally be analyzed to further characterize the detected ASFV. For example, if the first and second targets are detected, the assay result may be considered as positive for the wild type strain. If neither of the first or second targets are detected, but the generic target was detected, the assay result may be considered as positive for a double deletion, vaccine-associated variant. If one of the first or second targets is detected, but the other is not detected, the assay result may be considered as positive for another type of ASFV variant.

The test sample for the assays described herein may include or be derived from a variety of sources, including blood, serum, saliva, tissues, feces, urine, or environmental samples exposed or suspected of potential exposure to infected animals.

### Example Oligonucleotide Components

Embodiments disclosed herein include primers and optionally probes useful for the detection of targeted ASFV loci in a sample associated with an animal. Such primers and probes can be used in singleplex or multiplex nucleic acid assays, as described in more detail below, for detection and identification of the targets in a sample. The assays described herein demonstrate a high level of sensitivity, specificity, and accuracy. In some embodiments, the assay is designed to (1) detect the presence of ASFV in the sample, and (2) determine whether the ASFV is wild type ASFV or is a variant, such as a vaccine-associated variant.

In some embodiments, assays are configured to detect an amplification product of the target regions by detecting a signal from a label (i.e., a detectable label) or other signal-generating process, where the signal indicates formation of the amplification product. In some embodiments, the label is attached to, or otherwise associated with, the corresponding forward primer and/or reverse primer used to generate the amplification product. Additionally, or alternatively, the label is attached to, or otherwise associated with, a probe configured to associate with a probe binding sequence within the target region. In some embodiments, the label is an optically detectable label. Alternatively, the label may be detectable via non-optical means including electronically, electrically, or using NMR, sound, radioactivity, and the like.

In embodiments that include probes, the probes may be configured as TaqMan probes, which are known in the art and described in greater detail below. Such probes are able to hybridize to a target downstream from a primer such that exonuclease activity of the polymerase during subsequent primer extension separates a dye label from a quencher to increase the dye signal.

In some embodiments, the assay is multiplex and includes differentially labelled probes. For example, a first probe targeted to a first sample target (e.g., an MGF360 gene) may have a first label, while a second probe targeted to a second sample target (e.g., the CD2v gene) may have a second, different label. In some embodiments, a separate probe is associated with the generic target (e.g., the p72 gene) and includes a third label different from both the first and second labels. In other embodiments, the generic target is assayed in a separate reaction volume from the first and/or second targets, and therefore does not require a label that is different from both the first and second probes. In some embodiments, a separate probe is associated with an internal positive control (IPC) and includes a fourth label different from the first, second, and/or third labels. In other embodiments, however, the IPC is analyzed in a separate reaction volume from the first and/or second targets (e.g., with the generic target but not the first and second targets), and therefore does not require a label that is different from both the first and second probes.

Example primers and probes that may be used to detect the presence of the MGF360-14L target and the CD2v target are provided below in Table 1.

TABLE 1
Example Primers and Probes

| Target | SEQ ID NO. | Type | Sequence |
|---|---|---|---|
| MGF360- | SEQ ID | Forward | GAATAGGACCTAAATCCCACCACAA |
| 14L | NO: 1 | Primer | |
| | SEQ ID | Reverse | TGTCTTTACAAACGTTGGCCAAA |
| | NO: 2 | Primer | |
| | SEQ ID | Probe | ACCAAAACGCTGCAACGTATAAT |
| | NO: 3 | | CATAGTCAC |
| CD2v | SEQ ID | Forward | TGACACCACTTCCATACATGAACC |
| | NO: 4 | Primer | |
| | SEQ ID | Reverse | AGGGACGCATGTAGTAAATAGGTGTA |
| | NO: 5 | Primer | |
| | SEQ ID | Probe | TCTCCCAGAGAACCATTACTTCCTAA |
| | NO: 6 | | GCCT |

In one embodiment, the MGF360-14L probe is labelled with VIC, and the CD2v probe is labelled with FAM. However, these labels may be swapped, or other suitable labels, as known in the art and/or as described elsewhere herein, may be additionally or alternatively be utilized, including, but not limited to, JUN, ABY, Alexa Fluor dye labels (e.g., AF647 and AF676), and combinations thereof.

Assays may include the primer/probe sets for one or both targets shown in Table 1 to aid in determining whether detected ASFV is wild type or is a deletion variant type, such as a double deletion variant that would suggest it is a possible vaccine-associated variant. In some embodiments, one or both primer/probe sets shown in Table 1 may be combined with a primer/probe set configured to detect the presence of a generic ASFV target, such as the p72 region. The primer/probe set for the generic ASFV target may be combined with one or both primer/probe sets of Table 1 in a multiplex arrangement or as a separate component intended for use in a separate reaction volume.

As discussed above, detection of both the MGF360-14L and CD2v targets suggests that the tested sample includes wild type ASFV. Detection of neither of the MGF360-14L or CD2v targets (in conjunction with detection of a generic ASFV target such a p72) suggests that the tested sample includes a variant form of ASFV, such as a vaccine-associated variant. Detection of one of the MGF360-14L and CD2v targets suggests that the tested sample includes another variant form of ASFV.

### Example ASF Assay Kits

Example assay kits can include any of the primers and/or probes described herein, including the primer/probe sets of Table 1. In some embodiments, an assay kit further comprises a master mix. The primers and/or probes may be pre-mixed with and included as part of the master mix. The master mix may include, for example, a polymerase, nucleotides, one or more buffers, or one or more salts to promote amplification of the target when the mixture and a sample combined therewith are exposed to amplification conditions. In some embodiments, the primer/probe sets of Table 1 are combined with a master mix such as the TaqPath™ ProAmp™ Master Mix (Thermo Fisher Scientific, Catalog No. A30865) in a container.

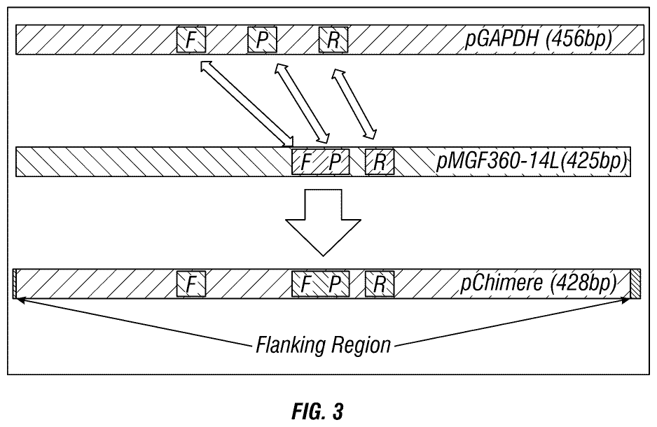

An assay kit as disclosed herein may also include an external positive control (EPC). The EPC may be provided in a container separate from the container holding the master mix and primer/probe sets. An example EPC is described in greater detail below with reference to FIG. 3 and Table 2.

In some embodiments, the assay kit includes an internal positive control (IPC), either pre-mixed with the master mix or provided in a separate container. However, in other embodiments, the assay kit excludes an IPC and is instead designed for use with a separate ASFV assay that targets a generic ASFV locus. The separate generic ASFV assay includes an IPC that can be leveraged to negate the need for a separate IPC within the variant ASFV assay. An example of such a generic ASFV assay is the VetMAX™ African Swine Fever Virus Detection Kit (Applied Biosystems, Catalog No. A28809).

In embodiments where the variant ASFV assay is combined with a generic ASFV assay, the combination of both assays may be referred to as a “combined ASFV assay” that includes both a “variant ASFV sub-assay” and a “generic ASFV sub-assay.” In some embodiments, both the variant ASFV sub-assay and the generic ASFV sub-assay include their own IPCs. In other embodiments, only one of the variant ASFV sub-assay or the generic ASFV sub-assay includes an IPC. In a presently preferred embodiment, the variant ASFV sub-assay omits an IPC, and the process flow is instead utilized in a manner that leverages the IPC of the generic ASFV sub-assay. Other embodiments may instead include an IPC for the variant ASFV sub-assay and not the generic ASFV sub-assay. In yet other embodiments, no IPC is included.

In some embodiments where the variant ASFV sub-assay omits an IPC and the generic ASFV sub-assay includes an IPC, both the variant ASFV sub-assay and the generic ASFV sub-assay may be designed as duplex assays. For example, in the variant ASFV sub-assay, a first probe with a first label may be associated with the first target (e.g., the MGF360-14L gene), and a second probe with a second, different label may be associated with the second target (e.g., the CD2v gene). In the generic ASFV sub-assay, a first probe with a first label may be associated with the generic target (e.g., the p72 gene), and a second probe with a second, different label may be associated with the IPC. The variant ASFV sub-assay may therefore be duplex with respect to two targeted loci where mutations/deletions are possible, whereas the generic ASFV sub-assay may be duplex with respect to the generic ASFV target and the IPC.

FIG. 2 illustrates an example of two separate process flows (i.e., methods) for using a combined ASFV assay that includes a generic ASFV sub-assay with IPC and a variant ASFV sub-assay without IPC. The “reagents” of the sub-assays refers to the respective master mixes of the sub-assays as described above (including respective primer/probe sets) in addition to any other desired components such as nuclease-free water, additional buffer, etcetera.

In the first process flow (top of FIG. 2), the test sample is mixed with the reagents of the generic ASFV sub-assay and the IPC in a first set of reaction volumes and with the reagents of the variant ASFV sub-assay in a second set of reaction volumes. The amplification reactions of each set of reaction volumes are carried out together. For example, as shown, the two sub-assays may be carried out together by dividing the wells of a well-plate such that a first set of wells include the test sample, the reagents of the generic ASFV sub-assay, and the IPC, while a second set of wells include the test sample and the reagents of the variant ASFV sub-assay. Alternative reaction volume arrangements may be utilized instead of or in addition to well plates. However, it is preferred that regardless of how the separate sets of reaction volumes are configured, the amplification reactions are carried out simultaneously or at least using the same instrument with substantially identical settings so that the IPC results of the generic ASFV reaction volumes remain applicable to the variant ASFV reaction volumes.

In the second process flow (bottom of FIG. 2), the generic ASFV sub-assay and the variant ASFV sub-assay are carried out sequentially. First, the test sample is mixed with the reagents of the generic ASFV sub-assay and the IPC. Amplification is then carried out. Only those wells in which the generic target (e.g., p72) is detected and in which the IPC is detected/validated are further analyzed using the variant ASFV sub-assay reagents to detect the presence of potential mutations/deletions (e.g., MGF360-14L and CD2v). In the illustrated second process flow, the first amplification reaction thus acts as a screening step to determine whether ASFV (in some form) is present before then further analyzing for the presence or absence of the variant-associated targets.

An assay kit as disclosed herein may also include an EPC. The EPC may be provided in a container separate from the container holding the master mix and primer/probe sets. In one example, an assay kit includes a first container comprising the primer/probe sets of Table 1 (or similar primer/probe sets for other suitable ASFV targets associated with a mutant/variant), and a second container comprising the EPC.

### Example External Positive Control

FIG. 3 illustrates an example of an EPC that may be utilized in conjunction with variant primer/probe sets (e.g., as in Table 1) in an assay for determining whether ASFV present in a sample is wild type ASFV or a variant ASFV. In this example, the EPC is based on a GAPDH plasmid modified to replace the GAPDH primer and probe regions with ASFV primer and probe regions that correspond to the primer/probe sets of the kit. The EPC plasmid is preferably designed such that the resulting amplicon length and distance between primers and probe substantially match the amplicon length and distance between primers and probe in the test sample reaction volumes. The EPC may include multiple inserts (e.g., one for the MGF360-14L target and one for the CD2v target). Alternatively, multiple plasmids, each with a single target, may together be utilized as the EPC.

Table 2 lists example insert sequences for the MGF360-14L and CD2v targets that may be incorporated into a plasmid (e.g., a GAPDH plasmid as in FIG. 3 or another suitable plasmid) to form an EPC suitable for use with an assay that includes the primer/probe sets of Table 1. The inserts may both be added to the same plasmid or may be singly added to separate plasmids that are then combined.

TABLE 2
Example EPC Insert Sequences

| | SEQ ID | Insert | |
|---|---|---|---|
| Target | NO. | Length | Sequence |
| MGF360- | SEQ ID | 442 bp | ATTGCTAATTGACCTTCACTACATGGTCTACATG |
| 14L | NO: 7 | | TTCCAGTATGATTCCACCCACGGCAAGTTCAACG |
| | | | GCACAGTCAAGGCAGAGAACGGGAAGCTCGTCA |
| | | | TCAATGGAAAGGCCATCACCATCTTCCAGGAGC |
| | | | GAGATCCTGCCAACATCAAGTGGGGTGATGCTG |
| | | | GTGCTGAGTATGTGGTGGAGTCCACTGGGGTCTT |
| | | | CACTACCATGGAGAAGGCTGGGGAATAGGACCT |
| | | | AAATCCCACCACAAACCAAAACGCTGCAACGTA |
| | | | TAATCATAGTCACAACACCCTCAAGATTGTCAG |
| | | | CAATGCCTTTTGGCCAACGTTTGTAAAGACAACT |
| | | | GCTTGGCCCCCCTGGCCAAGGTCATCCATGACC |
| | | | ACTTTGGCATCGTGGAGGGACTTATGACCACTGT |
| | | | CCACGCCATCACTGCCACCCAGAAGACTGTGGA |
| | | | TTGATTCA |
| CD2v | SEQ ID | 314 bp | ATTGCTAGGCAAGTTCAACGGCACAGTCAAGGC |
| | NO: 8 | | AGAGAACGGGAAGCTCGTCATCAATGGAAAGGC |
| | | | CATCACCATCTTCCAGGAGCGAGATCCTGCCAA |
| | | | CATCAAGTGTGACACCACTTCCATACATGAACCT |
| | | | TCTCCCAGAGAACCATTACTTCCTAAGCCTCAAG |
| | | | ATTGTCAGCAATGCCTTACACCTATTTACTACAT |
| | | | GCGTCCCTTGGCCCCCCTGGCCAAGGTCATCCAT |
| | | | GACCACTTTGGCATCGTGGAGGGACTTATGACC |
| | | | ACTGTCCACGCCATCACTGCCACCCAGAAGACT |
| | | | GTGGATTGATTCA |

### Additional Nucleic Acid Amplification & Detection Details

Amplified products resulting from use of one or more embodiments described herein can be generated, detected, and/or analyzed on any suitable platform. In some embodiments, the nucleic acid targets may be single-stranded, double-stranded, or any other nucleic acid molecule of any size or conformation. The amplification processes described herein can include PCR (see, e.g., U.S. Pat. No. 4,683,202). In some embodiments, the PCR is real time or quantitative PCR (qPCR). In some embodiments, the PCR is an end point PCR. In some embodiments, the PCR is digital PCR (dPCR). Other amplification methods, such as, e.g., loop-mediated isothermal amplification (“LAMP”), and other isothermal methods are also contemplated for use with the assay embodiments described herein.

Optionally, certain qPCR assays can be plated into individual wells of a single array or multi-well plate, such as for example a TaqMan Array Card (see, e.g., Thermo Fisher Scientific, Waltham, MA; Catalog Nos. 4346800 and 4342265) or a MicroAmp multi-well (e.g., 96-well, 384-well) reaction plate (see, e.g., Thermo Fisher Scientific, Waltham, MA; Catalog Nos. 4346906, 4366932, 4306737, 4326659 and N8010560). Optionally, the different qPCR assays present in different wells of an array or plate can be dried or freeze-dried in situ and the array or plate can be stored or shipped prior to use. In some embodiments, the concepts described herein may be utilized in in situ hybridization applications not necessarily associated with PCR.

In some embodiments, the primers described herein are used in nucleic acid assays at a concentration from about 100 nM to 1 mM (e.g., 300 nM, 400 nM, 500 nM, etc.), including all concentration amounts and ranges in between. In some embodiments, the probes described herein are used in a nucleic acid assay at a concentration from about 50 nM to 500 nM (e.g., 75 nM, 125 nM, 250 nM, etc.), including all concentration amounts and ranges in between.

The primers and/or probes described herein may further comprise a fluorescent or other detectable label. In some embodiments the primers and/or probes may further comprise a quencher and in other embodiments the probes may further comprise a minor groove binder (MGB) moiety. Suitable fluorescent labels include but are not limited to 6FAM, ABY, VIC, JUN, and FAM. Suitable quenchers include but are not limited to QSY (e.g., QSY7 and QSY21), BHQ (Black Hole Quencher) and DFQ (Dark Fluorescent Quencher).

The primer and probe sequences described herein need not have 100% homology/identity to their targets to be effective, though in some embodiments, homology is substantially 100% or exactly 100%. In some embodiments, one or more of the disclosed primer and/or probe sequences have a homology to their respective target of at least about 50%, at least about 60%, at least about 70%, at least about 75%, at least about 80%, at least about 85%, at least about 90%, at least about 95%, at least about 97%, at least about 98%, at least about 99%, at least about 99.9%, or up to substantially 100% or exactly 100%. Some combinations of primers and/or probes may include primers and/or probes each with different homologies to their respective targets, and the homologies may be, for example, within a range with endpoints defined by any two of the foregoing values.

In some embodiments, different assay products can be independently detected or at least discriminated from each other. For example, different assay products may be distinguished optically (e.g., using optically different labels for each qPCR assay) or can be discriminated using some other suitable method, including as described in U.S. Patent Publication No. 2019/0002963, which is incorporated herein by reference in its entirety.

The reaction vessel or volume can optionally include a tube, channel, well, cavity, site or feature on a surface, or alternatively a droplet (e.g., a microdroplet or nanodroplet) that may be deposited onto a surface or into a surface well or cavity, or suspended within (or partially bounded by) a fluid stream. In some embodiments, the reaction volume includes one or more droplets arrayed on a surface or present in an emulsion. The reaction volumes can optionally be formed by fusion of multiple pre-reaction volumes containing different components of an amplification reaction. For example, pre-reaction volumes containing one or more primers can be fused with pre-reaction volumes containing human nucleic acid samples and/or polymerase enzymes, nucleotides, and buffer. In some embodiments involving performing qPCR reactions in array format, a surface contains multiple grooves, channels, wells, cavities, sites, or features defining a reaction volume containing one or more amplification reagents (e.g., primers, probes, buffer, polymerase, nucleotides, and the like). In some array-formatted singleplex embodiments, the reaction volume within the selected tubes, grooves, channels, wells, cavities, sites, or features contains only a single forward primer sequence and a single reverse primer sequence. Optionally, one or more probe sequences are also included in the singleplex reaction volume.

Assays described herein may utilize TaqMan probes. TaqMan-based assays are typically carried out by performing nucleic acid amplification on a target polynucleotide using a nucleic acid polymerase having 5′-to-3′ nuclease activity. The probe typically includes a detectable label (e.g., a fluorescent reporter molecule) and a quencher molecule capable of quenching the fluorescence of the reporter molecule. Typically, the detectable label and quencher molecule are part of a single probe. As amplification proceeds, the polymerase digests the probe to separate the detectable label from the quencher molecule. The detectable label is monitored during the reaction, where detection of the label corresponds to the occurrence of nucleic acid amplification (e.g., the higher the signal the greater the amount of amplification). Variations of TaqMan assays are known in the art and would be suitable for use in the methods described herein.

Other detectable labels may be used in addition to or as an alternative to labelled probes. For example, primers can be labeled and used to both generate amplicons and to detect the presence (or concentration) of amplicons generated in the reaction, and such may be used in addition to or as an alternative to labeled probes described herein. As a further example, primers may be labeled and utilized as described in Nazarenko et al. (Nucleic Acids Res. 2002 May 1; 30 (9): e37), Hayashi et al. (Nucleic Acids Res. 1989 May 11; 17 (9): 3605), and/or Neilan et al. (Nucleic Acids Res. Vol. 25, Issue 14, 1 Jul. 1997, pp. 2938-39). Those of skill in the art will also understand and be capable of utilizing the PCR processes (and associated probe and primer design techniques) described in Zhu et al. (Biotechniques. 2020 July: 10.2144/btn-2020-0057).

Any of these systems and detectable labels, as well as many others, may be used to detect amplified target nucleic acids. In some embodiments, intercalating labels can be used such as ethidium bromide, SYBR Green I, SYBR GreenER, and PicoGreen (Life Technologies Corp., Carlsbad, CA), thereby allowing visualization in real-time, or end point, of an amplification product in the absence of a detector probe. In some embodiments, real-time visualization may include both an intercalating detector probe and a sequence-based detector probe. In some embodiments, the detector probe is at least partially quenched when not hybridized to a complementary sequence in the amplification reaction and is at least partially unquenched when hybridized to a complementary sequence in the amplification reaction. In some embodiments, probes may further comprise various modifications such as a minor groove binder to further provide desirable thermodynamic characteristics.

In some embodiments, the amplicon is labeled by incorporation of or hybridization to labeled primer. In some embodiments, the amplicon is labeled by hybridization to a labeled probe. In some embodiments, the amplicon is labeled by binding of a DNA-binding dye. In some embodiments, the dye may be a single-strand DNA binding dye. In other embodiments, the dye may be a double-stranded DNA binding dye. In other embodiments, the amplicon is labeled via polymerization or incorporation of labeled nucleotides in a template-dependent (or template-independent) polymerization reaction. This can be part of the amplifying step or alternatively the labeled nucleotide can be added after amplifying is completed. The labeled amplicon (or labeled derivative thereof) can be detected using any suitable method such as, for example, electrophoresis, hybridization-based detection (e.g., microarray, molecular beacons, and the like), chromatography, NMR, and the like.

In one exemplary embodiment, the labeled amplicon is detected using capillary electrophoresis. In another embodiment, the labeled amplicon is detected using qPCR. In some embodiments, a plurality of different amplicons is formed, and optionally labeled, within a single reaction volume via a single amplification reaction. For example, a multiplex reaction (e.g., 2-plex, 3-plex, 4-plex, 5-plex, 6-plex) carried out in a single tube or reaction vessel (e.g., “single-tube” or “1-tube” or “single-vessel” reaction) can produce a plurality of amplicons that are labeled. In some embodiments, the plurality of amplicons can be differentially labeled. In some embodiments, each of the plurality of amplicons produced during amplification is labeled with a different label.

In some embodiments, the nucleic acid amplification assays as described herein are performed using a Real-time PCR (qPCR) instrument, including for example a QuantStudio Real-Time PCR system, such as the QuantStudio 5 RealTime PCR System (QS5), QuantStudio 7 RealTime PCR System (QS7), and/or QuantStudio 12K Flex System (QS12K), or a 7500 Real-Time PCR system, such as the 7500 Fast Dx system, from Thermo Fisher Scientific.

### Additional Terms & Definitions

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described embodiment, any of the possible alternatives listed for that element or component may generally be used individually or in combination with one another, unless implicitly or explicitly stated otherwise.

In addition, unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, less than 1%, less than 0.1%, or less than 0.01% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

It will also be appreciated that embodiments described herein may also include properties and/or features (e.g., ingredients, components, members, elements, parts, and/or portions) described in one or more separate embodiments and are not necessarily limited strictly to the features expressly described for that particular embodiment. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

1. A composition for the detection of African swine fever virus (ASFV) in a test sample, the composition comprising:
a first primer set configured to enable amplification of a first target location including at least a portion of an MGF360 gene; and/or
a second primer set configured to enable amplification of a second target location including at least a portion of the CD2v gene.

2. The composition of claim 1, wherein the MGF360 gene is MFG360-14L.

3. The composition of claim 1 or claim 2, wherein the first primer set comprises one or both of SEQ ID NO:1 or SEQ ID NO:2 for enabling amplification of the first target location.

4. The composition of any one of claims 1-3, wherein the second primer set comprises one or both of SEQ ID NO:5 or SEQ ID NO:6 for enabling amplification of the second target location.

5. The composition of any one of claims 1-4, further comprising a first probe with a first label, the first probe configured to hybridize to a first amplicon resulting from the first primer set.

6. The composition of claim 5, wherein the first probe comprises or is SEQ ID NO:3.

7. The composition of claim 5 or claim 6, further comprising a second probe with a second label, the second probe configured to hybridize to a second amplicon resulting from the second primer set.

8. The composition of claim 7, wherein the second probe comprises or is SEQ ID NO:6.

9. The composition of claim 7 or claim 8, wherein the first label and the second label are different.

10. The composition of any one of claims 1-9, further comprising an additional primer set configured to enabler amplification of an additional ASFV locus.

11. The composition of claim 10, wherein the additional ASFV locus comprises at least a portion of the p72 gene.

12. The composition of claim 10 or claim 11, further comprising an additional labelled probe configured to hybridize to an amplicon resulting from the additional primer set.

13. An assay kit for the detection of African swine fever virus (ASFV) in a test sample, the kit comprising a master mix that includes:
a composition as in any one of claims 1-9; and
optionally, one or more of a polymerase, nucleotides, one or more buffers, or one or more salts.

14. The kit of claim 13, further comprising an external positive control (EPC).

15. The kit of claim 14, wherein the master mix and the EPC are provided in separate containers.

16. The kit of claim 14 or claim 15, wherein the EPC is a plasmid with one or more inserts corresponding to (i) an MGF360 gene, or portion thereof, and/or (ii) the CD2v gene, or portion thereof.

17. A combination assay kit for the detection of African swine fever virus (ASFV) in a test sample and for distinguishing between wild type ASFV and variant strains of ASFV, the kit comprising:
a variant ASFV sub-assay comprising a kit as in any one of claims 13-16 for the detection of the MGF360 and/or CD2v targets; and
a generic ASFV sub-assay configured to detect one or more generic ASFV targets.

18. The kit of claim 17, wherein the one or more generic ASFV targets includes the p72 gene or a portion thereof.

19. The kit of claim 17 or claim 18, further comprising an internal positive control (IPC).

20. The kit of claim 19, wherein the IPC is included in the generic ASFV sub-assay and wherein the variant ASFV sub-assay omits an IPC.

21. The kit of claim 20, wherein the variant ASFV sub-assay is a multiplex assay having a first label associated with detection of the MGF360 target and a second, different label associated with detection of the CD2v target.

22. The kit of claim 20 or claim 21, wherein the generic ASFV sub-assay is a multiplex assay having a first label associated with detection of the one or more generic ASFV targets and a second, different label associated with detection of the IPC.

23. A method of detecting one or more African swine fever virus (ASFV) targets in a test sample and optionally characterizing detected ASFV as wild type or a variant type, the method comprising:
providing a test sample comprising or suspected of comprising ASFV;
mixing the test sample with a composition as in any one of claims 1-12 to form a reaction mixture;
subjecting the reaction mixture to amplification conditions; and
detecting presence or absence of the MGF360 target and/or CD2v target in the sample.

24. The method of claim 23, further comprising determining that the test sample comprises or is likely to comprise wild type ASFV when both the MGF360 target and the CD2v target are detected.

25. The method of claim 23 or claim 24, further comprising detecting the presence or absence in the test sample of one or more generic ASFV targets.

26. The method of claim 25, further comprising determining that the test sample comprises or is likely to comprise a variant ASFV when (i) the one or more generic ASFV targets are detected, and (ii) at least one of the MGF360 target or the CD2v target are not detected.

27. The method of claim 25, wherein when neither of the MGF360 target or the CD2v target are detected, the ASFV is identified as a double deletion variant.

28. The method of claim 27, wherein the double deletion variant is further identified as a vaccine-associated variant.

29. A method of detecting one or more African swine fever virus (ASFV) targets in a test sample and characterizing detected ASFV as wild type or a variant type, the method comprising:
providing a test sample comprising or suspected of comprising ASFV;
providing a combination assay that comprises
a variant ASFV sub-assay configured to detect one or more variant-associated ASFV targets, and
a generic ASFV sub-assay configured to detect one or more generic ASFV targets;
forming reaction mixtures in separate reaction volumes associated with the variant ASFV sub-assay and the generic ASFV sub-assay, respectively;
subjecting the reaction mixtures to amplification conditions;
detecting the presence or absence of the one or more generic ASFV targets in the reaction volume associated with the generic ASFV sub-assay; and
detecting the presence or absence of the one or more variant-associated ASFV targets in the reaction volume associated with the variant ASFV sub-assay.

30. The method of claim 29, further comprising determining that the test sample comprises or is likely to comprise wild type ASFV when the one or more generic ASFV targets are detected in the reaction volume associated with the generic ASFV sub-assay and the one or more variant-associated ASFV targets are detected in the reaction volume associated with the variant ASFV sub-assay.

31. The method of claim 29 or claim 30, further comprising determining that the test sample comprises or is likely to comprise a variant ASFV when the one or more generic ASFV targets are detected in the reaction volume associated with the generic ASFV sub-assay but one or more variant-associated ASFV targets are not detected in the reaction volume associated with the variant ASFV sub-assay.

32. The method of any one of claims 29-31, wherein the one or more generic ASFV targets includes the p72 gene or portion thereof.

33. The method of any one of claims 29-32, wherein the one or more variant-associated ASFV targets includes one or both of (i) an MGF360 gene, or portion thereof, or (ii) the CD2v gene, or portion thereof.

34. The method of claim 33, wherein when neither the MGF360 or CD2v targets are detected, the ASFV is identified as a double deletion variant.

35. The method of claim 34, wherein the double deletion variant is further identified as a vaccine-associated variant.

36. The method of any one of claims 29-35, wherein the variant ASFV sub-assay and the generic ASFV sub-assay are provided as a kit as in any one of claims 17-22.

37. The method of any one of claims 29-36, wherein the generic ASFV sub-assay includes an IPC and wherein the variant ASFV sub-assay omits an IPC.

38. The method of claim 37, wherein subjecting the reaction mixtures to amplification conditions comprises simultaneously subjecting reaction mixtures associated with the variant ASFV sub-assay and the generic ASFV sub-assay to the same amplification conditions.

39. The method of claim 37, wherein subjecting the reaction mixtures to amplification conditions comprises first subjecting reaction mixtures associated with the generic ASFV sub-assay, including the IPC, to amplification conditions, then subsequently forming one or more reaction mixtures associated with the variant ASFV sub-assay by mixing with only those reaction mixtures in which the one or more generic ASFV targets and the IPC are detected.


SEQUENCE LISTING (8 sequences)

SEQ ID NO: 1 - DNA, 25 bp, synthetic construct
  source 1..25: mol_type = other DNA
gaataggacctaaatcccaccacaa

SEQ ID NO: 2 - DNA, 23 bp, synthetic construct
  source 1..23: mol_type = other DNA
tgtctttacaaacgttggccaaa

SEQ ID NO: 3 - DNA, 32 bp, synthetic construct
  source 1..32: mol_type = other DNA
accaaaacgctgcaacgtataatcatagtcac

SEQ ID NO: 4 - DNA, 24 bp, synthetic construct
  source 1..24: mol_type = other DNA
tgacaccacttccatacatgaacc

SEQ ID NO: 5 - DNA, 26 bp, synthetic construct
  source 1..26: mol_type = other DNA
agggacgcatgtagtaaataggtgta

SEQ ID NO: 6 - DNA, 30 bp, synthetic construct
  source 1..30: mol_type = other DNA
tctcccagagaaccattacttcctaagcct

SEQ ID NO: 7 - DNA, 442 bp, synthetic construct
  source 1..442: mol_type = other DNA
attgctaattgaccttcactacatggtctacatgttccagtatgattccacccacggcaagttcaacggcacagtcaaggcagagaacgggaagctcgtcatcaatggaaaggccatcaccatcttccaggagcgagatcctgccaacatcaagtggggtgatgctggtgctgagtatgtggtggagtccactggggtcttcactaccatggagaaggctggggaataggacctaaatcccaccacaaaccaaaacgctgcaacgtataatcatagtcacaacaccctcaagattgtcagcaatgccttttggccaacgtttgtaaagacaactgcttggcccccctggccaaggtcatccatgaccactttggcatcgtggagggacttatgaccactgtccacgccatcactgccacccagaagactgtggattgattca

SEQ ID NO: 8 - DNA, 314 bp, synthetic construct
  source 1..314: mol_type = other DNA
attgctaggcaagttcaacggcacagtcaaggcagagaacgggaagctcgtcatcaatggaaaggccatcaccatcttccaggagcgagatcctgccaacatcaagtgtgacaccacttccatacatgaaccttctcccagagaaccattacttcctaagcctcaagattgtcagcaatgccttacacctatttactacatgcgtcccttggcccccctggccaaggtcatccatgaccactttggcatcgtggagggacttatgaccactgtccacgccatcactgccacccagaagactgtggattgattca